(19) United States
(12) Patent Application Publication
(10) Pub. No.: US 2015/0164785 A1
(43) Pub. Date: Jun. 18, 2015

(54) DEVICES AND METHODS FOR TREATING THE GASTROINTESTINAL SYSTEM

(71) Applicant: Bridgepoint Medical Inc., Plymouth, MN (US)

(72) Inventors: Chad J. Kugler, Andover, MN (US); Robert E. Atkinson, White Bear Lake, MN (US); Peter T. Keith, St. Paul, MN (US)

(21) Appl. No.: 14/570,972

(22) Filed: Dec. 15, 2014

Related U.S. Application Data
(63) Continuation of application No. 11/408,238, filed on Apr. 21, 2006.
(60) Provisional application No. 60/674,323, filed on Apr. 22, 2005.

(51) Int. Cl. A61K 9/00; A61K 31/78; A61K 33/00; A61K 31/765; A61K 31/785; A61K 31/75; A61K 31/745

(52) U.S. Cl. CPC A61K 9/0012; A61K 31/75; A61K 31/78; A61K 31/745; A61K 31/765; A61K 31/785; A61K 33/00

(57) ABSTRACT

Devices and methods for less invasively treating the gastrointestinal system, such as the intestinal mucosa of the small bowel. Embodiments described herein may be used to reduce caloric absorption and effectuate weight loss, for example.

This application claims the benefit of U.S. Provisional Application No. 60/674,323, filed Apr. 22, 2005, under 35 U.S.C. §119(e). The entire disclosure of that provisional application is incorporated by reference herein.

### FIELD OF THE INVENTION

The embodiments described herein generally relate to devices and methods to treat the gastrointestinal system. For example, some embodiments described herein relate to the treatment of obesity and/or its associated co-morbidities.

### BACKGROUND OF THE INVENTION

A variety of devices and methods have been proposed for the treatment of obese patients, particularly morbidly obese patients, in an effort to achieve significant weight loss. These therapeutic methods may be generally characterized as restrictive and/or malabsorptive.

Restrictive therapies typically involve reducing gastric volume (e.g., forming a small gastric pouch) and/for reducing gastric outflow size (e.g., forming a narrow stoma) such that the patient achieves a sensation of satiety with less consumed food. Examples of surgical techniques for restrictive therapy include gastric reduction (“stomach stapling”) and vertical banded gastroplasty (VBG). An example of a less invasive technique for restrictive therapy is laparoscopic adjustable gastric banding (LAGB, e.g., Lap Band™). However, restrictive therapy, taken alone, has been met with limited clinical success.

Malabsorptive therapies typically involve removing, diverting or bypassing a portion of the intestinal path such that the patient absorbs less of the food consumed. Some of the more effective gastric bypass operations, such as Roux-en-Y gastric bypass, also involve the formation of a small gastric pouch and/or narrow stoma, and therefore may be characterized as both restrictive and malabsorptive. Despite the effectiveness of some combined restrictive and malabsorptive therapies, they still involve significant surgery to reroute, rearrange and reconnect portions of the intestinal path, and are therefore often associated with significant complications.

### SUMMARY OF THE INVENTION

To address these issues and other unmet needs, embodiments of the present invention provide, by way of example, not limitation, less invasive devices and methods for treating the gastrointestinal system, such as the intestinal mucosa of the small bowel to reduce absorption. The devices and methods described herein may be used to effectuate weight loss by compromising caloric absorption to thereby treat obesity and its associated co-morbidities, for example. However, it is contemplated that the devices and methods described herein may be used to treat other adverse health conditions associated with the gastrointestinal system but not specifically mentioned herein. Furthermore, the devices and methods described herein may be used alone or in combination with other devices and methods used to facilitate gastrointestinal therapy.

### BRIEF DESCRIPTION OF THE DRAWINGS

It is to be understood that both the foregoing summary and the following detailed description are exemplary. Together with the following detailed description, the drawings illustrate exemplary embodiments and serve to explain certain principles. In the drawings,

FIG. 1 is an anatomical illustration showing a three dimensional section of the wall of the jejunum taken through a circular plication;

FIG. 2 is an anatomical illustration showing a two dimensional cross section of the intestinal wall taken through a villus;

FIG. 3 is a schematic block diagram illustrating the flow of nutrients from the intestinal lumen to the vascular system via the intestinal mucosa;

FIG. 4A illustrates an orally administered capsule or pill to deliver the desired therapy;

FIGS. 4B(1) and 4B(2) illustrate a fixed diameter capsule;

FIGS. 4C(1) and 4C(2) illustrate an expandable diameter capsule;

FIG. 5A illustrates a transesophageally endoscopically delivered catheter for purposes of administering the desired therapy;

FIG. 5B illustrates a transesophageally or endoscopically delivered and peristaltic directed catheter for purposes of administering the desired therapy;

FIG. 6A illustrates a laparoscopically inserted intraintestinal catheter for purposes of administering the desired therapy;

FIG. 6B illustrates a surgically inserted intraintestinal catheter for purposes of administering the desired therapy;

FIGS. 7A and 7B illustrate means for activating and deactivating an orally administered therapeutic capsule;

FIGS. 7C-7G illustrate various patterns of therapeutic application to the mucosal surface of the intestine;

FIG. 8A illustrates mucosal viii of the intestine covered by a coating;

FIGS. 8B(1) and 8B(2) illustrate a capsule for coating or paving the intestinal villi;

FIGS. 9A and 9B illustrate a Peltier effect capsule for thermal effect on the intestinal mucosa;

FIGS. 10A and 10B illustrate an RF capsule for thermal effect on the intestinal mucosa;

FIG. 11 illustrates an endothermic or exothermic capsule for thermal effect on the intestinal mucosa;

FIG. 12 illustrates a resistive heating capsule for thermal effect on the intestinal mucosa;

FIG. 13 illustrates a microwave capsule for thermal effect on the intestinal mucosa;

FIGS. 14A and 14B illustrate a radiation capsule for radioactive effect on the intestinal mucosa;

FIGS. 15A and 15B illustrate an ultrasonic capsule for cavitation effect on the intestinal mucosa;

FIGS. 16A-16G illustrate chemical eluting capsules for chemical effect on the intestinal mucosa;

FIGS. 17A-17D illustrate a PDT capsule for microvascular effect on the intestinal mucosa; and

FIG. 18 illustrates an infusion catheter for local delivery of micro-occlusive agent for microvascular effect on the intestinal mucosa.

### DETAILED DESCRIPTION OF EXEMPLARY EMBODIMENTS

The following detailed description should be read with reference to the drawings in which similar elements in different drawings are numbered the same. The drawings, which are not necessarily to scale, depict illustrative embodiments and are not intended to limit the scope of the invention.

To better understand certain principles of the inventions described herein, a brief anatomical description fellows. The gastrointestinal tract generally includes the stomach, the small intestine and the large intestine. The small intestine is a long (approximately 20 ft.) convoluted tubular structure that follows a circuitous path between the stomach and the large intestine. The small intestine is segmented into three parts: the duodenum (approximately 10 inches long); the jejunum (approximately 7½ ft. long); and the ileum (approximately 11½ ft, long). The duodenum receives partially digested food contents emptied from the stomach and receives secretions from the bile and pancreatic ducts. The jejunum extends from the duodenum to the ileum, and the ileum extends from the jejunum to the colon (large intestine).

The primary digestive functions of the small intestine are (1) to facilitate further chemical digestion of food, and (2) to facilitate absorption of nutrients into the blood stream. The duodenum is primarily involved in secretion (mucus, enzymes and hormones) and chemical (enzymatic) digestion. The jejunum is primarily involved in nutrient absorption, and employs an intestinal wall specially adapted for that function. The ileum is also involved in nutrient absorption, but primarily involves water absorption. As such, the devices and methods described herein may specifically target all or a portion of the jejunum (distal of the duodenum and proximal of the ileum) to compromise caloric nutrient absorption without compromising the bile and pancreatic ducts, and without compromising the secretion and enzymatic digestion of the duodenum, and without compromising water absorption of the ileum or colon.

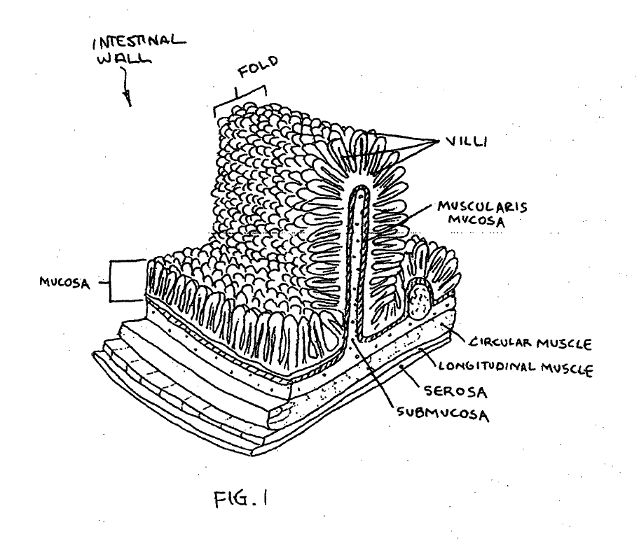

With reference to FIG. 1, a three dimensional section of the wall of the jejunum is shown in perspective view. The mucosal (inner) surface of the jejunum is defined by numerous circular folds or placations covered by a plethora of villi. Each villus is covered by a layer of absorptive epithelial cells, each of which includes a plurality of micro-villi extending therefrom. The collective surface area provided by the folds, the villi and the micro-villi in the small intestine is approximately 3200 sq. ft., or more than 450 times that provided by a smooth intestinal wall of similar dimension, which facilitates the absorptive function thereof. The circular folds and villi are much more prevalent in the jejunum than in other parts of the small intestine, and thus the primary digestive function of the jejunum is nutrient absorption.

With continued reference to FIG. 1, the mucosal (inner) surface of the intestinal wall may be defined to include the epithelial layer, the villous layer (villi), and the muscularis mucosa layer. The epithelial layer and villous layer (villi) are primarily responsible for nutrient absorption and transport into the microvasculature, and the muscularis mucosa layer is responsible for motility of the villi. Immediately below the mucosal layer is the submucosa, which is surrounded by two external muscularis layers including the circular muscularis and the longitudinal muscularis. The external muscularis layers are responsible for gross motility or peristaltic motion of the intestinal wall, and are surrounded by the serosa which forms the outer surface of the intestine.

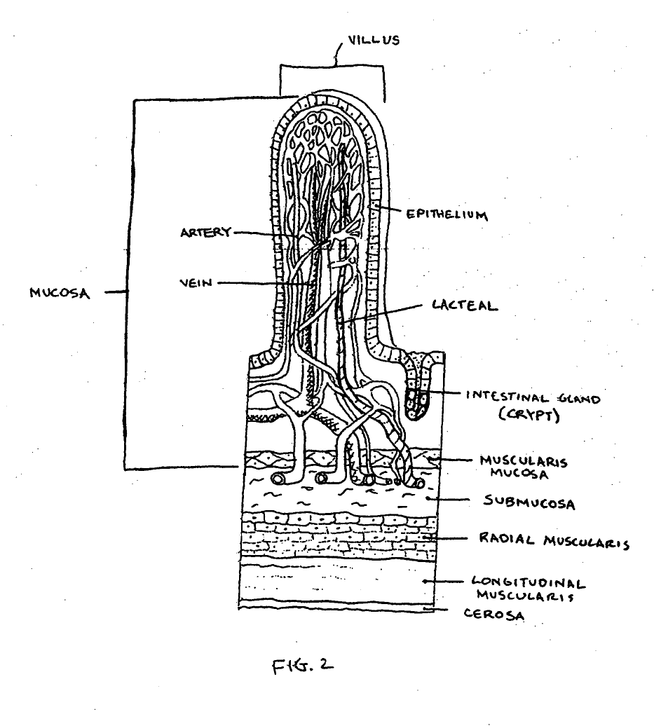

With reference to FIG. 2, a two dimensional cross section of the intestinal wall taken through a villus is shown. Each villus is covered by epithelium comprising closely packed absorptive epithelial cells having micro-villi extending therefrom. At the base of the villi are intestinal glands (crypts or ducts) lined by absorptive cells and endocrine secretion cells. The epithelial cells at the apex of the viii are sloughed of or destroyed (e.g., due to abrasive damage) and regularly replaced by regeneration of the cells at the base of the villi in the crypts.

With continued reference to FIG. 2, the lamina propria or internal structure of the villi includes arterial and venous microvascular components (with their associated capillary bed) as well as terminal (blind or dead-end) lymph vessels or lacteals. Also contained within the lamina propria are smooth muscle cells and nerves. Generally speaking, the venous microvasculature carries away water soluble nutrients, and the lacteals carry away fat soluble nutrients. The venous microvasculature coalesces to form venules which follow a venous network leading to the hepatic portal vein and liver. The lacteals coalesce into lymph vessels and lymph nodes.

By these structures, digested nutrients may be absorbed into the vascular system of the body. As used herein, and depending on the particular context, absorption may include passive diffusion, facilitated diffusion, active transport, and/or co-transport from the intestinal lumen and into the vasculature. Generally, the epithelial layer is primarily involved in nutrient absorption, and the lamina propria is primarily involved in nutrient transport. As such, some embodiments of the present invention may affect or target the epithelial layer, some embodiments may affect or target the lamina propria, and some embodiments may affect or target both. In this context, the embodiments described herein generally compromise the ability of the gastrointestinal system to absorb nutrients. For example, absorption of caloric nutrients may be compromised to effectuate weight loss in order to treat and/or prevent adverse health conditions associated therewith and/or in order to achieve cosmetic effect.

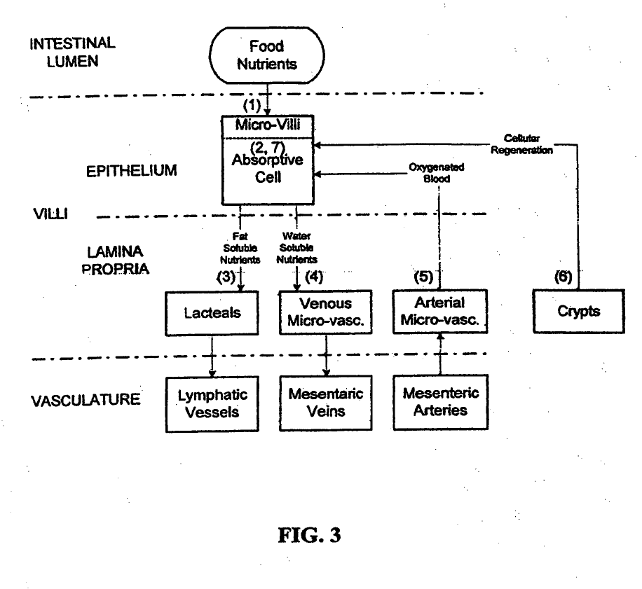

With reference to FIG. 3, a schematic block diagram is shown which illustrates the flow of nutrients from the intestinal lumen to the vascular system via the intestinal mucosa. The schematic diagram shown in FIG. 3 is generalized for purposes of illustrating certain characteristics of the embodiments described herein, and is not intended to be physiologically precise in infinite detail.

As shown in FIG. 3, nutrients from digested food in the intestinal lumen are absorbed into the viii through the epithelial layer comprising absorptive cells having micro villi thereon. Generally, fat soluble nutrients are absorbed by the lacteals in the lamina propria of the villi, and carried away via lymphatic vessels. Also generally, water soluble nutrients are absorbed by the venous microvasculature in the lamina propria of the villi, and carried away by the mesenteric veins leading to the portal vein and the liver. Oxygenated blood provides nutrients to the villi structures via the mesenteric arteries and the arterial microvasculature in the lamina propria. Absorptive epithelial cells are regenerated at the base of the villi in the crypts and migrate up the villi.

Generally, the embodiments described herein may be characterized in terms of how they affect the physiological absorption process, or in terms of how they affect the anatomical structures associated therewith. The former is described in more detail with reference to FIG. 3, and the later is described in more detail thereafter.

With continued reference to FIG. 3, there are a number of points in the generalized absorption process that may be compromised to cause malabsorption. By way of example, not limitation, the absorption process may be compromised by (1) shielding the epithelial layer from exposure to food nutrients in the intestinal lumen; (2) compromising the diffusion or transport mechanism of the absorptive cells; (3) compromising the transport of (fat soluble) nutrients through the lacteals; (4) compromising the transport of (water soluble) nutrients through the venous microvasculature; (5) compromising the transport of oxygenated blood through the arterial microvasculature to render the villi dysfunctional; (6) compromising the regeneration of absorptive cells by the crypts; or (7) causing premature necrosis or sloughing of the absorptive cells. While the net effect of interfering with the absorption process according to the preceding methods may be malabsorption, the intervening effects may include dysfunctional or slow absorption mechanism(s), compromised integrity or functionality of mucosal structures (e.g., villi, lamina propria, epithelium, and/or crypts), reduced surface area available for absorption, etc.

Specific examples of embodiments for compromising the absorptive process at these points are described in more detail hereinafter. These embodiments include, but are not limited to, coating the mucosal surface and compromise of the mucosa structures by thermal, chemical, mechanical vibratory (e.g., ultrasonic), radioactive radiation, microvascular restriction, and/or lacteal restriction means.

These embodiments involve delivery by a variety of different approaches including, but not limited to, transesophageal, endoscopic, laparoscopic, and surgical approaches. For example, delivery may be accomplished by an orally administered capsule 40, an endoscopically delivered catheter 50, a laparoscopically inserted intraintestinal catheter 60A, and/or a surgically inserted intraintestinal catheter 60B. Prior to delivery, a gastrointestinal flush may be administered to provide clear access to the mucosal surfaces.

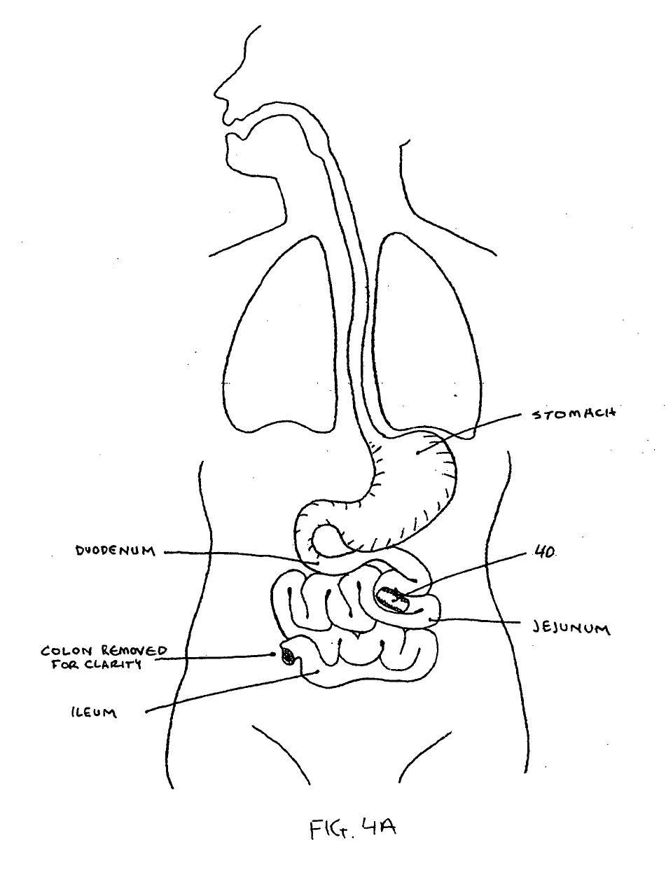

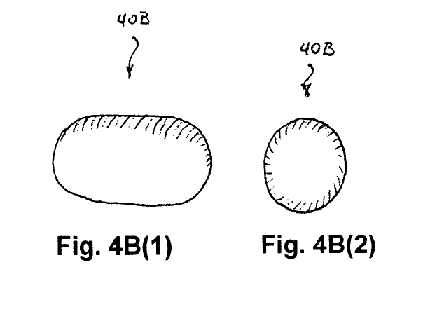

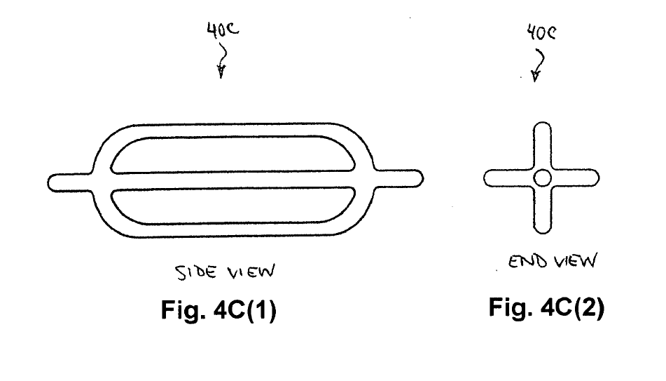

FIG. 4A illustrates an orally administered capsule or pill 40A that is sized and shaped to atraumatically travel through the gastrointestinal tract utilizing natural peristaltic motion of the tract to facilitate passage therethrough. The capsule may be swallowed in a conventional manner, traveling through the oral cavity, down the esophagus and into the gastric cavity (stomach). From the stomach, the capsule travels into the small intestine, through the large intestine (colon), and is expelled out the sphincter. The pill may include a tether (similar to the catheters described hereinafter) extending proximally therefrom for purposes of supplying electrical power, radioactive seeds, coating agents, hot or cold liquids, chemicals or other liquids to the intestinal site.

The capsule 40 may have a tapered and rounded profile with a fixed diameter (40B) as shown in FIG. 4B(1) (side view) and 4B(2) (end view) or an expandable diameter (40C) as shown in FIGS. 4C(1) (side view) and 4C(2) (end view). The expandable embodiment may have a plurality of collapsible portions that radially expand such that the capsule may be collapsed in narrow portions of the gastrointestinal tract (e.g., a sphincter or stoma), and expand in larger portions thereof, enabling intimate contact with the mucosal surfaces independent of luminal size. The expandable capsule may be self-expandable utilizing a super elastic nickel titanium alloy structure, for example, or actuatable utilizing a magnetically triggered spring biased mechanism, for example. An example of a mechanism for expansion includes several longitudinal members which bow outwards after a restrictive membrane (not shown) dissolves after a period of time after the capsule is swallowed. One or more of the longitudinal members may contain one or more of the various therapy means (e.g., chemical eluting ports, ultrasonic transducers, RF electrodes, radiation elements or windows, thermal contacts, etc.) described in more detail hereinafter.

FIG. 5A illustrates a catheter 50 delivered through the oral cavity and esophagus and into the gastrointestinal tract. The catheter may be delivered alone, over a guide wire (not shown) and/or through an endoscope (not shown) to facilitate navigation. The catheter may have a length sufficient to extend through the oral cavity, esophagus, and gastric cavity and into the desired location in the intestinal tract according to the anatomical dimensions described previously. The catheter may comprise a polymeric tubular structure, and may be reinforced with a metallic coil or braid to improve pushability and reduce kinkability, and may include one or more lumens extending therethrough to facilitate delivery of fluids or other devices therein. The proximal end of the catheter 50 may be connected to a pressurized fluid source 52 such as a syringe for infusion of fluids. FIG. 5B illustrates a similar catheter 50 but with a modified tip 54 to aid in advancement through the tortuous gastrointestinal tract. The modified tip may have an enlarged bulbous shape, for example, which is biased distally by peristaltic motion of the gastrointestinal tract and/or by the flow of gastrointestinal contents therethrough.

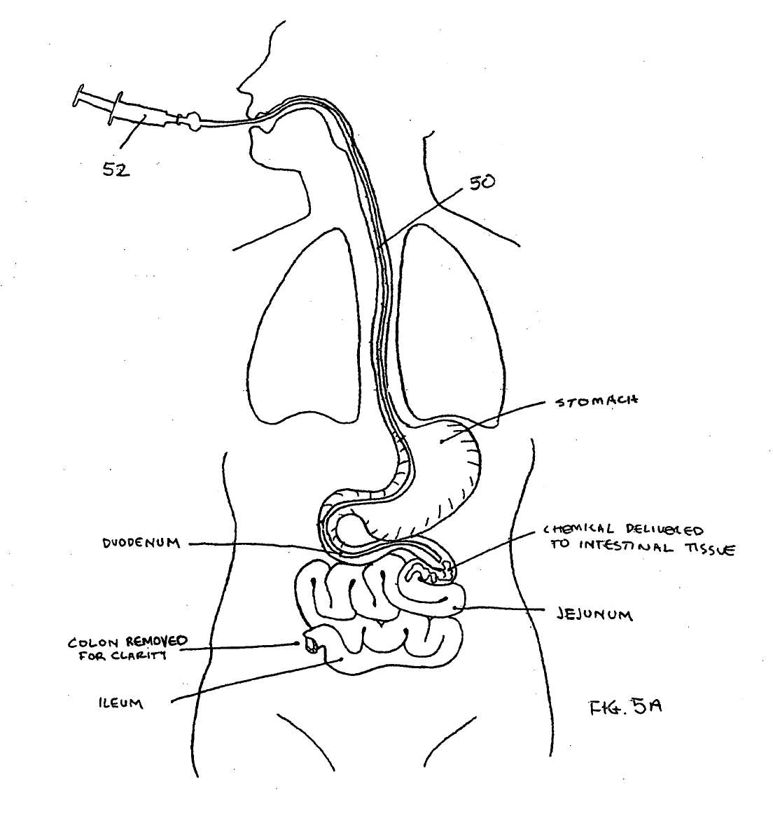

FIG. 6A illustrates a catheter 60A similar to the catheter illustrated in FIG. 5A, but introduced laparoscopically through a laparoscope, for example, and inserted into a portion of the gastrointestinal tract such as the small intestine as shown. The catheter may be delivered over a stylet (not shown) or similar device to facilitate penetration into the gastric or intestinal wall. FIG. 6B illustrates a similar catheter 60B but inserted surgically through the abdominal wall and into a portion of the gastrointestinal tract such as the small intestine as shown.

The various embodiments may specifically target the small intestine or a portion thereof such as the duodenum, jejunum, and or ileum. Because the primary function of the jejunum is absorption, it may be desirable to target the jejunum or a portion thereof, to avoid compromising the pancreatic and bile secretions of the duodenum. However, it is contemplated that the therapy provided hereby may be applied to any portion of the gastrointestinal tract to have the desired effect.

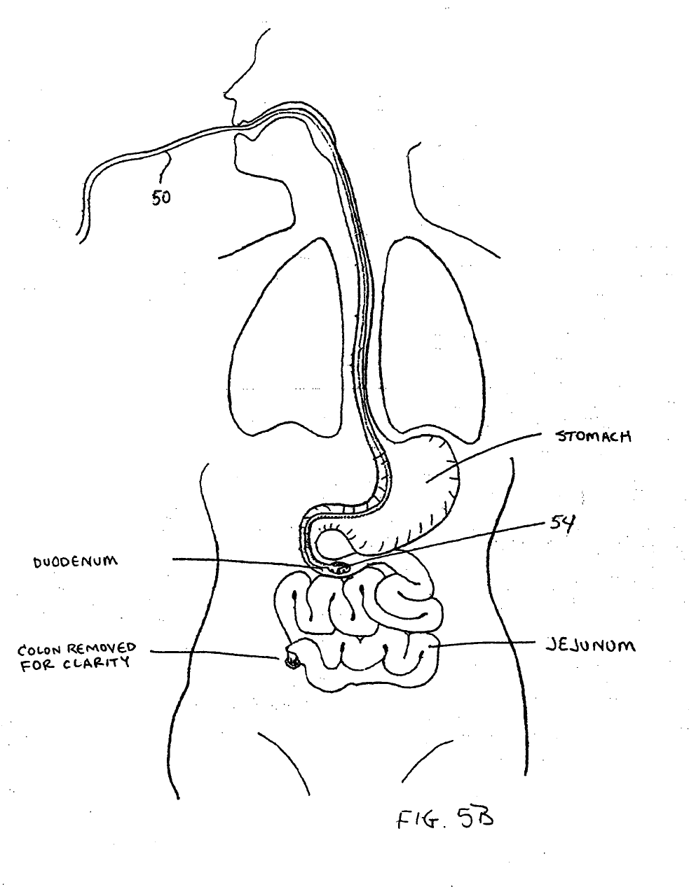

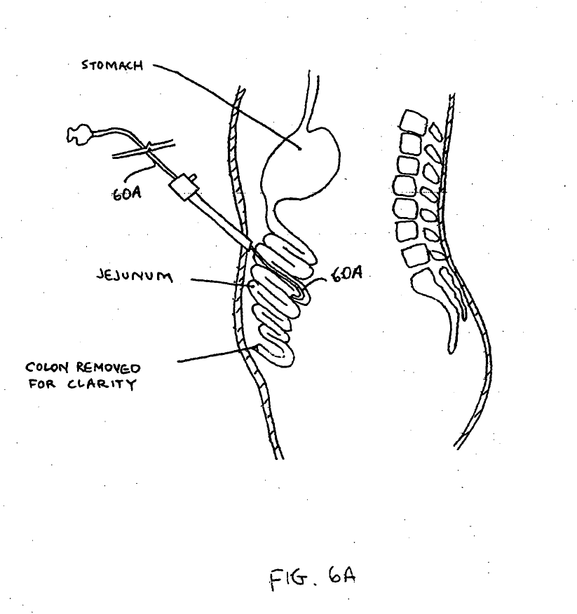

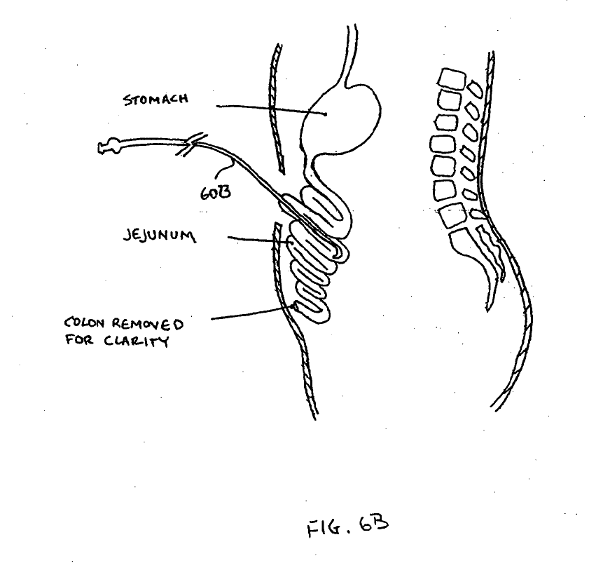

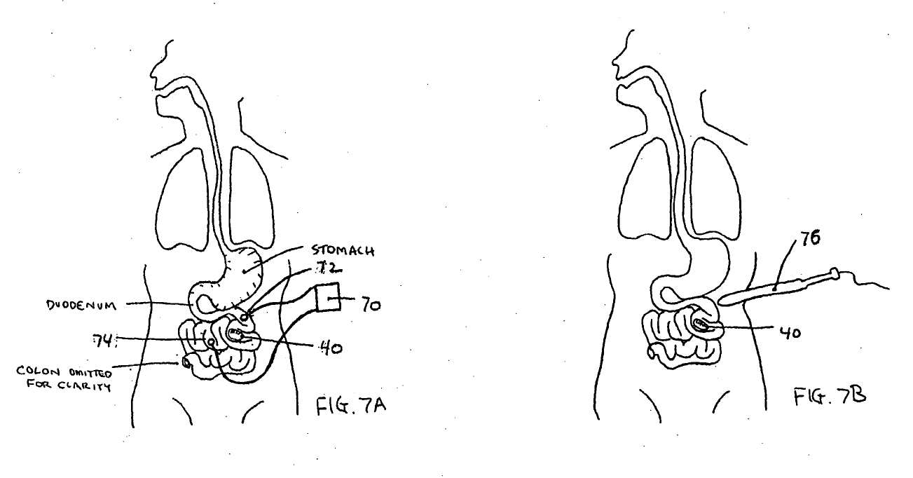

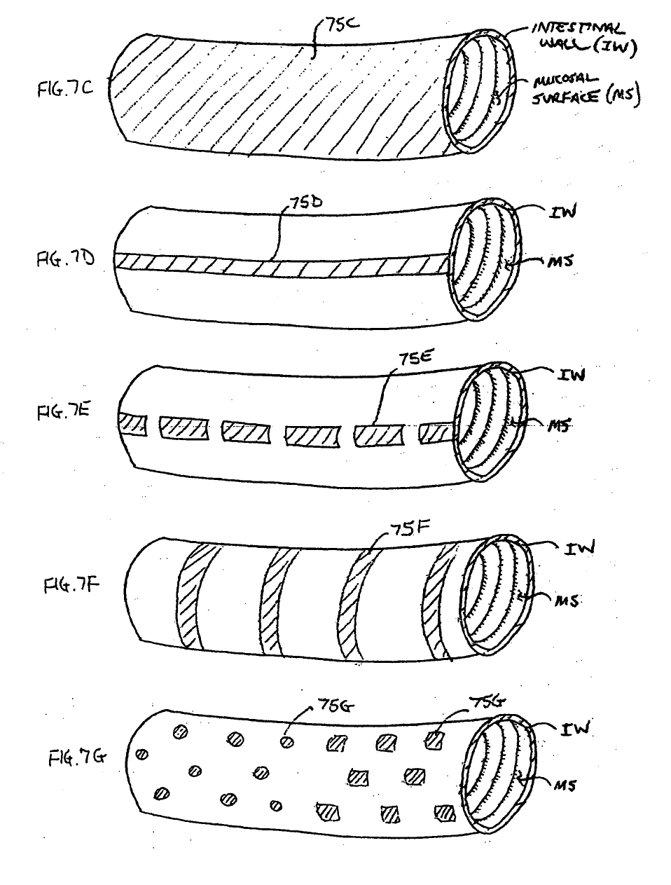

In order to target a portion of the small intestine, for example, a capsule may be turned on and off by detecting the position of the capsule by utilizing radiofrequency or ultrasonic transmission and tracking techniques as shown in FIG. 7A, or by non-invasive visualization techniques as shown in FIG. 7B. These embodiments provide the ability to dimensionally control the treatment initiation, the length of intestine to be treated, and the treatment cessation.

For example, with reference to FIG. 7A, the control may be accomplished by telemetrically communicating with the capsule 40 through the use of one or more external transmitting, receiving or transceiving devices that communicate with a corresponding device in the capsule though magnetic, radiofrequency, or ultrasonic fields. In the illustrated embodiment, two or more separate emitters are connected to a control unit 70 placed on the skin at predetermined locations in close proximity to the intestine where the treatment will start and stop. The treatment will initiate as the capsule passes by the first emitter 72 and cease as it passes by the second emitter 74. Other emitters may be used to create multiple start and stop points or to change the treatment intensity.

Alternatively, with reference to FIG. 7B, a hand held wand 76 may be used initiate the treatment. Similar to the previous embodiment, the wand may contain a transmitting, receiving or transceiving device that communicates with a corresponding device in the capsule 40 though magnetic, radiofrequency, or ultrasonic fields. With the aid of visualization via x-ray, magnetic resonance imaging, or other suitable technique, the capsule may be monitored for position with respect to the small intestine. Once the capsule reaches the targeted area through intestinal peristalsis, the wand is passed over the patient's abdomen to turn the capsule on and initiate treatment. Monitoring of the capsule may continue until it reaches its distal most treatment site. The wand is passed over the patient's abdomen again to turn the capsule off to complete the treatment. The rate of intestinal peristalsis may be increased by the administration of a diuretic.

In addition, the therapies provided herein may be applied to all or a portion of the targeted anatomical structure. For example, with reference to FIGS. 7C-7G, the therapy may be applied to the entire mucosal surface (full radial and full longitudinal coverage 75C) as seen in FIG. 7C. Alternatively, it may be applied as a longitudinal stripe or helix 75D (partial radial and full longitudinal) as seen in FIG. 7D, a dashed longitudinal stripe or helix 75E (partial radial and partial longitudinal) as seen in FIG. 7E, or a series of rings 75F (full radial and partial longitudinal) as seen in FIG. 7F. Any of the foregoing may be used standing alone or in combination with each other, and may be applied in any of a variety of patterns and shapes, such as those 75G shown in FIG. 7G.

One embodiment for compromising the absorptive process involves coating, covering or otherwise physically blocking the mucosal surface from exposure to nutrients as shown in FIG. 8A. For example, portions of the intestinal mucosa may be blocked by selectively coating 80 the interior surface of thereof with an appropriate agent to prevent nutrients from reaching the absorptive structures of the mucosa. The coating 80 may cover the top of the villi or infuse between the villi as shown in FIG. 8A.

FIGS. 8B(1) (side view) and 8B(2) (end view) illustrate a capsule 82 for delivery of a coating substance at desired locations within the gastrointestinal tract. The capsule may contain one or more ports 84, one or more reservoirs 86, and a pumping mechanism 88. In the illustrated embodiment, the pumping mechanism is a piston which pressurizes the coating agent (in liquid form), causing it to flow toward and out of the ports. A power source (not shown) and a motor for driving the piston (not shown) are also included inside the capsule. Administration of the coating may be controlled as by turning the pumping mechanism of the capsule on and off as described previously. Also as described previously, the coating may be applied in “strips”, or in “spots”. It is believed that “spots” will have less impact on intestinal motility, particularly with coating materials that are relatively rigid.

Several polymeric agents are suitable for use to coat the intestinal lumen, including epoxy resin, fluoropolymer, phenolic resin, melamine resin, polyacetal, polyacetylene, polyacrylic, polyalkylene, polyalkenylene, polyamic acid, polyamide, polyamine, polyanhydride, polyarylene, polybenzyl, polycarbodiiumide, polycarbonate, polycarbosilane, polycarborane, polydiene, polyester, polyurethane, polyetherketone, polyether, polyimidazole, polyimine, polyimide, polyisocyanate, polyisocyanurate, polyketone, polyolefin, polyoxide, polyoxyalkylene, polyoxyarylene, polyphenyl, polyquinoline, polysilane, polysiloxane, polyurea, polyvinylacetal, polyacetal, polysaccharide, and cyanoacrylate. These polymers, once cured, resist absorption by the intestine, and will withstand the pH environment therein.

Cyanocrylates are typically cured in the presence of moisture, which is abundant on the intestinal surface. The capsule may be coated with a hydrophilic coating to prevent the capsule from sticking to the mucosa as the coating is applied. Cyanoacrylates do not require the use of a separate curing agent, and it is contemplated that they may be applied directly from the ports of the capsule, either continuously during transit, or may be intermittently applied from the ports either automatically, or through the control means described previously. With the use of cyanoacrylates, only one reservoir and pump may be necessary, coupled to one or more ports in the capsule.

For coating materials that require a separate curing agent, such as epoxy, a separate reservoir for the curing agent may provided. FIGS. 8B(1) and 8B(2) show such an embodiment, wherein the distal (right) reservoir would contain the resin, and the proximal reservoir (left) would contain the curing agent. The resin may be delivered to the luminal surface, and as the capsule travels downstream, the curing agent would be delivered to the resin. Alternately, a mixing chamber within the capsule could be incorporated to mix the resin and the curing agent prior to deliver out of the ports.

The capsule may also contain a reservoir and one or more ports to deliver a preparatory agent, such as a primer, to render the intestinal lining more “bondable” to the coating. For example, a solvent could be delivered from the distal most ports to help break down the mucous layer of the mucosa, enabling the coating to better adhere to the intestinal lining.

Other embodiments for compromising the absorptive process involve thermally (hot or cold) treating the mucosal surface. Generally, these embodiments elevate or reduce the mucosal tissue temperature above or below a threshold temperature to cause cellular damage. These embodiments include, but are not limited to, a resistance heating device, an electrical device capable of producing the Peltier effect, an exothermic or endothermic chemical reaction and a radio frequency energy device.

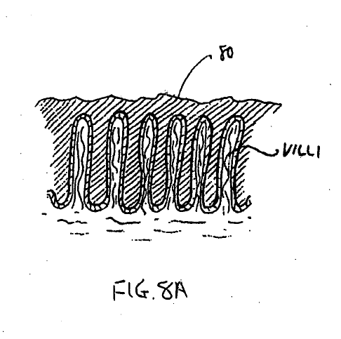

In one thermal embodiment, as shown in FIGS. 9A (side cross-sectional view) and 9B (end cross-sectional view), an electrical device 90 is placed inside a capsule 40 that uses the Peltier effect to heat or cool mucosal tissues. This device utilizes a power source 92 and a conduction loop of alternating semiconductors 94(A&B) interconnected by conductors 96 to generate a surface of increased temperature on one side of the device and a surface of reduced temperature on the opposing side. This circuit is known as a Peltier module to those skilled in the art. Acceptable semiconductor materials include but are not limited to iron and constantan, copper and nickel, and lead and constantan. Reversing the electrical polarity also reverses the position of hot and cold on the Peltier module thus changing the temperature profile on the surface of the capsule. An integrated circuit may be added to regulate, control polarity, or pulse the temperature. Multiple arrays of Peltier modules may be used both radially and longitudinally to create many different temperature patterns on the surface of the capsule resulting in different treatment geometries.

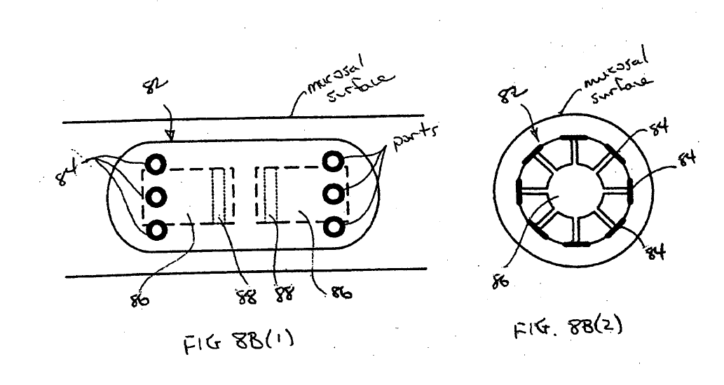

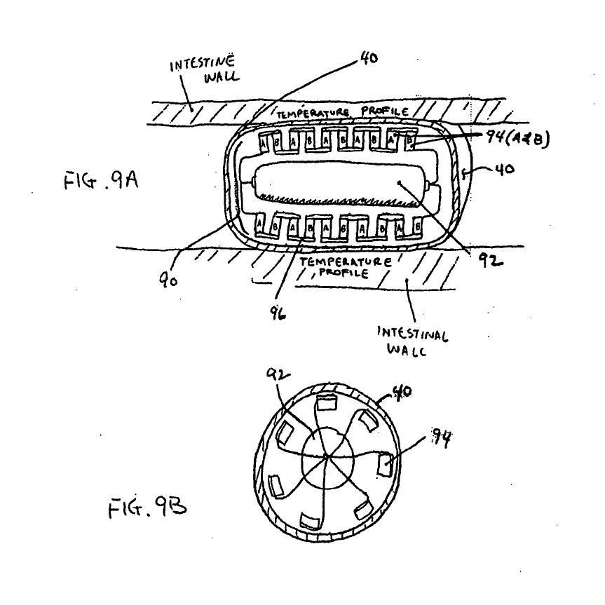

In another thermal embodiment, as shown in FIGS. 10A (side cross-sectional view) and 10B (end cross-sectional view), an electrical device 100 capable of delivering radio frequency energy to a conductor on the outside surface of the capsule. The capsule includes a power source 102 and a signal generator 104 capable of creating a radiofrequency energy wave. One or multiple arrays of contacts 106 (interconnected by conductors 108) may be used to contact the tissue may be used both radially and longitudinally. The signal generator may also have the ability to generate pulses of R.F. energy and also control when the device is turned on or off.

In another thermal embodiment, as shown in FIG. 11 (side cross-sectional view), two or more chemicals 110 (A&B) are placed inside the capsule 40 that generate an endothermic or exothermic reaction. The chemicals may be separated by one or more bladders 112 until mixing is desired. Examples of suitable endothermic reactants include but are not limited to citric acid+sodium bicarbonate, and barium hydroxide octahydrate+ammonium nitrate. Examples of suitable exothermic reactants include but are not limited to calcium oxide+water and calcium dichloride+water. A release mechanism 114 has the ability to rupture the bladders through electrical or electromechanical means. This release mechanism may be activated through telemetry from outside of the patient, for example.

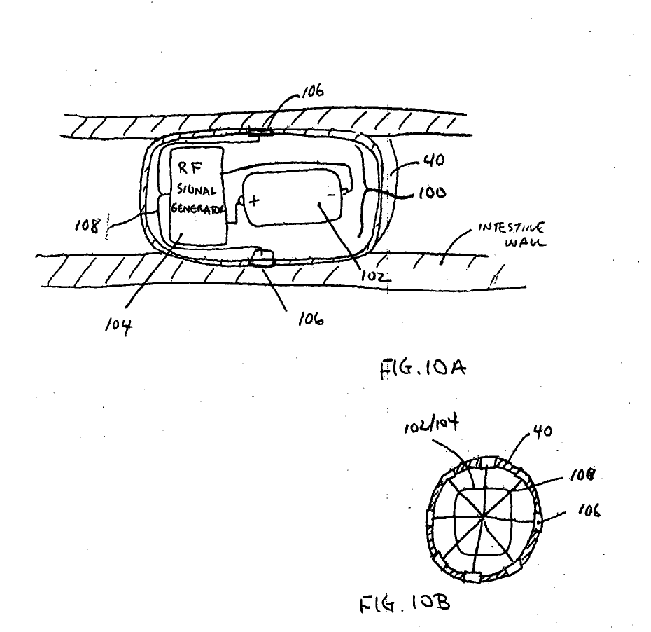

In another thermal embodiment, as shown in FIG. 12 (side cross-sectional view), a capsule 40 contains an electrical device 120 capable of heating intestinal tissues through one or more resistance heating elements 122 positioned on the outside of the capsule. The capsule also contains a power source 124 a control circuit 126 capable of turning the power to the heating elements on and off. One or more arrays of heating elements may be arranged both radially and longitudinally to contact in the desired pattern.

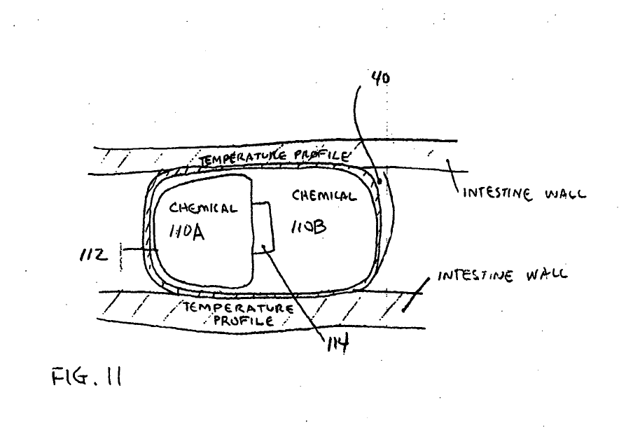

In another thermal embodiment, an electrical device 130 capable of delivering microwave energy is disposed in a capsule 40 to heat mucosal tissues as shown in FIG. 13 (side cross-sectional view). The capsule houses a power source 132 and a generator 134 capable of creating microwave emissions. The microwave generator may have the ability to generate pulses of microwave energy and may be turned on and off as described previously. The capsule may include one or more transmitting antennae and one or more receiving antennae to limit create localized transmission “loops” in order to limit depth penetration, thereby treating the mucosal structures while avoiding damage to the submucosa. The microwave emissions cause oscillation of water based molecules which are prevalent on the mucosal surface, thereby causing injury thereto and compromising nutrient absorption.

Other embodiments for compromising the absorptive process involve the use of radioactive radiation selectively exposed to the mucosal surface to cause injury or necrosis to a limited depth. While it is contemplated that all forms of radiation (alpha, beta, and gamma) could be used, beta radiation is advantageous because it is readily attenuated by bodily tissue and therefore has limited depth penetration, thus providing exposure to the mucosal layer while minimizing trauma to the submucosal tissues. Devices incorporating beta emitters are also more easily transported and handled, requiring less shielding than gamma emitters.

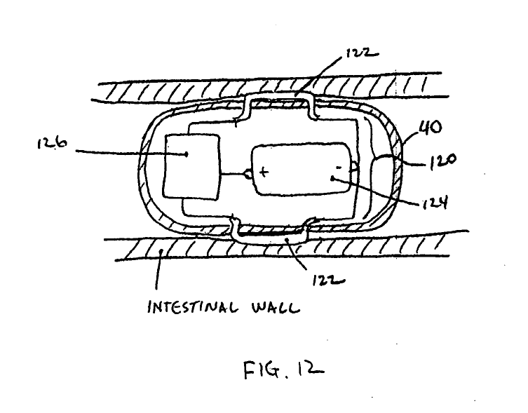

Radiation may delivered to the luminal surface of the intestine with a capsule 40 containing a beta emitter 140, as illustrated in FIGS. 14(A) (side view) and 14(B) (end view). The capsule is preferably rounded at both ends, and at one or more discrete locations therein contains an alloy of a radioactive substance 140. The capsule housing 142 shields radiation and includes one or more windows 144 through which radiation passes. The windows may include a retractable shield (not shown) to selectively expose the mucosa to radiation using the control means described previously. Phosphorus-32, Vanadium 48, and Yttrium-90 are three examples of beta-emitting elements which could be alloyed into the radioactive substance.

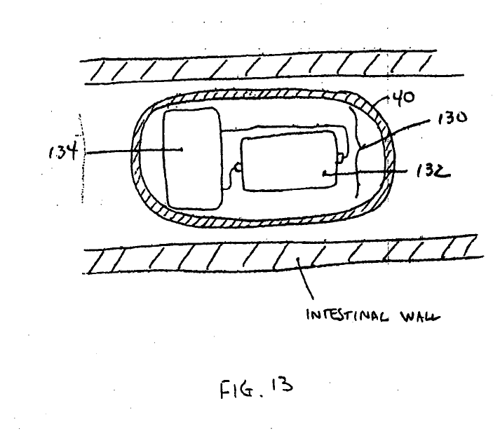

Other embodiments for compromising the absorptive process involve ablation of the intestinal mucosa with the use of ultrasonic energy as illustrated in FIGS. 15A (side view) and 15B (end view). The capsule 40 may include one or more ultrasonic transducers 150 powered by a control module 152 and battery 154. The transducers may extend around the entire periphery of the capsule or a portion thereof, depending on the pattern of ablation desired. In addition, the transducers may be selectively turned on and off utilizing the control means described previously. The transducers, when powered, emit ultrasonic waves which cause any liquid proximate the transducers to cavitate, forming small micro bubbles. When the micro bubbles collapse in the vicinity of the intestinal lining, they cause mechanical trauma, thus injuring or ablating the lining.

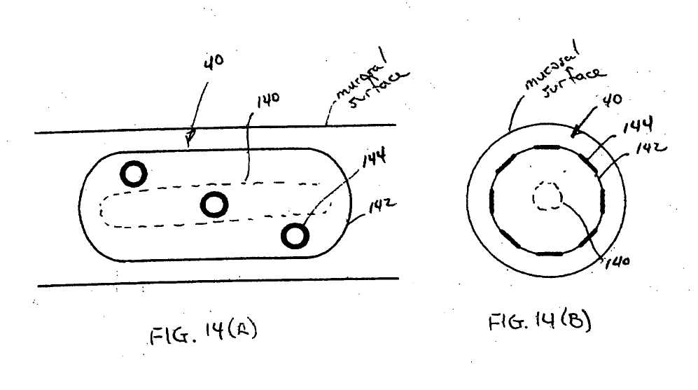

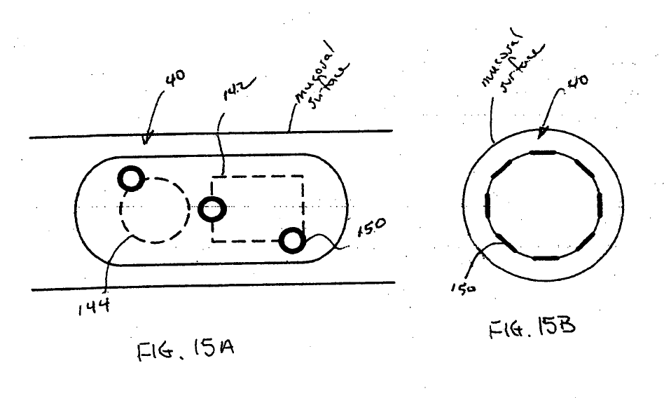

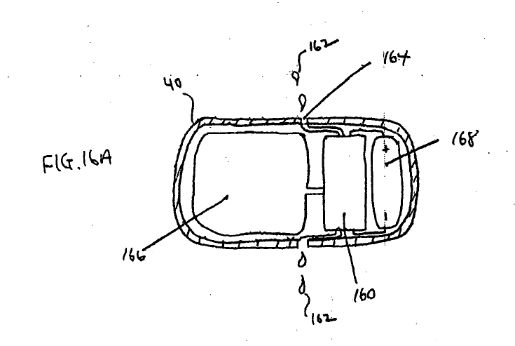

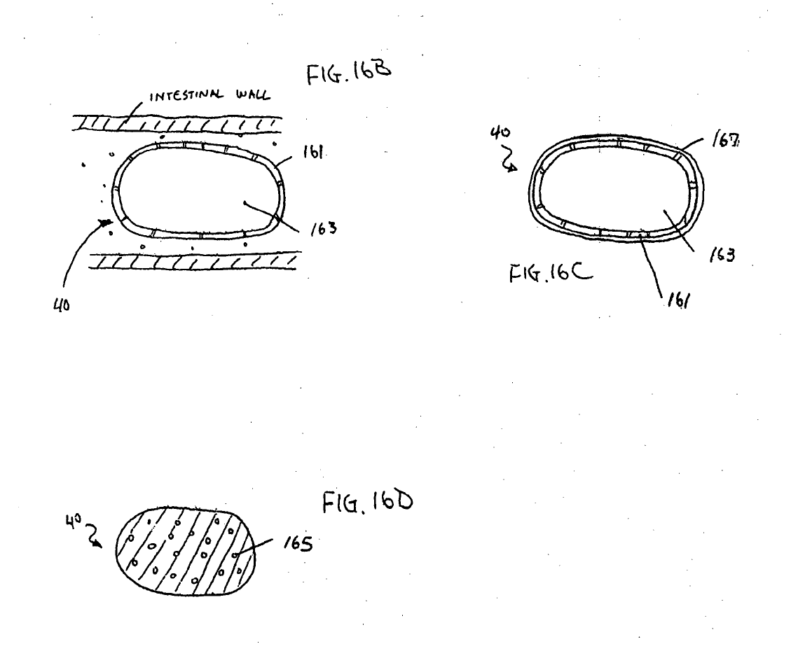

Other embodiments for compromising the absorptive process involve chemical injury to the mucosal surface. For example, in the embodiment illustrated in FIG. 16A, a micro pump 160 is placed inside a capsule 40 that dispenses a chemical 162. This battery 168 powered pump transports the chemical from a bladder 166 inside the capsule through one or more ports 164 in the capsule's exterior shell. Examples of suitable chemicals in diluted form include but are not limited to acetic acid, ethanol, benzalkonium chloride, and hydrochloric acid.

In another embodiment using chemical means to cause injury, a capsule 40 includes a porous shell 161 around a bolus of liquid chemical 163 as shown in FIG. 16B. The porosity of the shell allows the chemical to escape through diffusion at a desired rate. An alternative embodiment as shown in FIG. 16C places a biodesolvable or biodegradeable non-porous coating 167 over the aforementioned porous coating to delay chemical release. This coating would allow the capsule to be swallowed and would delay the chemical release until the capsule reached the level of the small intestine. Another alternative embodiment as shown in FIG. 16D uses a porous biodisolvable or biodegradable material 165 where the open interstices are filled with a liquid chemical. Breakdown of the material releases the chemical from the pores. Porous, bio-stable materials may include but are not limited to poly urethanes, silicones, polymethyl methacrylate, poly vinyl alcohol, polyethylene, polyacrylic acid. Biodegradable or biodisolvable materials may include but are not limited to polylactides (PLA), polyglycolides (PGA), polylactic-co-glycolic acid copolymer (PLGA), polyanhydrides, and polyorthoesters.

In yet another embodiment using chemical means to cause injury, an ingestible capsule 40 as shown in FIG. 16E (anatomical view) may be employed to dissolve when exposed to digestive fluids and release one or more active agents that will effect intestinal tissues and render them unable to absorb nutrients. As shown in FIGS. 16F (side cross-sectional view) and 16G (end cross-sectional view), the capsule 40 may also have one or more layers designed to control agent release with respect to anatomic position or release an agent designed to neutralize or dilute the active agent. For example, the capsule may have an outer layer 162 designed to allow it to traverse the esophagus, stomach and duodenum before dissolving and exposing underlying layers 164 containing the active agents (position #1). Layers may also be designed to dissolve at different rates to control the active agent release and concentration in a given length of intestine or to control the desired length of treatment. Potential active agents may include but are not limited to ethanol, benzalkonium chloride, acetic acid, and hydrochloric acid. A neutralizing or diluting agent 166 may be disposed inside the active agent or subsequently delivered in capsule or liquid form (e.g., by another capsule or ingested drink) to ensure termination of treatment (position #2). Neutralizing or diluting agents include but are not limited to an ingestible antacid or water.

Any of the embodiments using chemical means to cause injury may alternatively employ a catheter as described previously to administer the chemical agent by local infusion of the active agent, neutralizing agent and/or diluting agent to the intestinal mucosa.

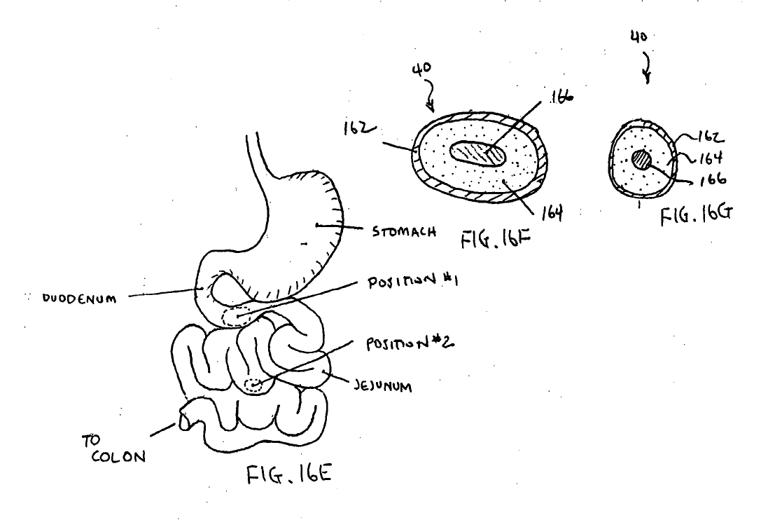

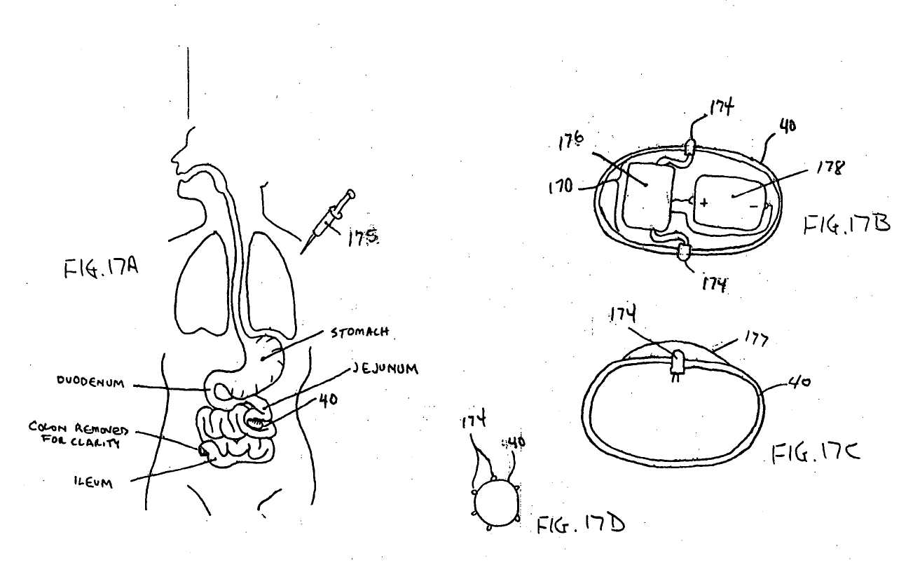

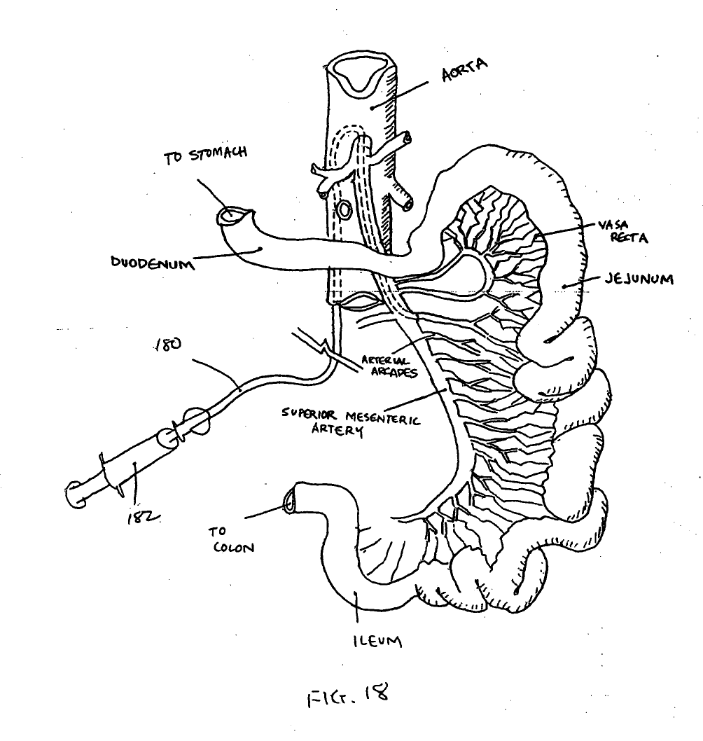

Other embodiments for compromising the absorptive process involve a combination of a photo-dynamic drug and a capsule 40 with a light emitting device 170 therein which are used to thrombose the intestinal microvasculature and render the mucosal tissues unable to absorb nutrients. The photodynamic drug may be administered intravenously (e.g., by injection using syringe 175) as shown in FIG. 17A (anatomical view). The light emitting capsule, with appropriate intensity and wavelength or color to activate the photodynamic drug, may be delivered trans-orally to the level of the small intestine. The combination of systemically administered photodynamic drug and site specific delivery of light allows the targeted occlusion of the intestinal microvasculature. In one embodiment, the light emitting device 170 may comprise one or more light emitting diodes 174 together with an associated control circuit 176 and power source 178 as shown in FIG. 17B. Another embodiment includes a diffuser 177 to increase the area of intense light as shown in FIG. 17C. Multiple arrays of lights may be used radially or longitudinally to achieve the desired pattern of effect on the mucosal surface as shown in FIG. 17D.

Yet another embodiment for compromising the absorptive process involves inhibiting the absorption of the mucosa by occluding the micro-vasculature supplying or internal to the intestinal villi as shown in FIG. 18. An infusion catheter 180 is introduced via femoral, subclavian, or radial access and advance through the ostium of the superior mesenteric artery using techniques known to those skilled in the art. The catheter is further advanced to the level of the arterial arcades. The distal end of the infusion catheter is positioned in the ostium of an artery that feeds a section of the intestine targeted for treatment.

A solution of occlusion beads designed to freely pass through the larger arteries of the arcades and the vasa recta and aggregate in the micro vasculature of the intestinal villi are introduced (e.g., using a syringe 182) via the infusion catheter. The micro bead materials may include but are not limited to suitable polymeric materials such as polyethylene, polymethyl methacrylate, anadine butadiene styrene, polycarbonate, polyamide, and pebax. Other materials may include ceramic, glass, and metal. The beads may be suspended in a solution of aqueous saline for ease of delivery from a syringe 182.

Bead aggregation in the microvasculature results in occlusion of the oxygenated blood supply to the villi. This occlusion will cause intestinal tissue necrosis and also ceases the needed blood circulation needed for nutrient absorption and transport.

From the foregoing, it will be apparent to those skilled in the art that the present invention provides, in exemplary non-limiting embodiments, devices and methods for less invasively treating the gastrointestinal system, such as the intestinal mucosa of the small bowel to reduce caloric absorption and effectuate weight loss. Further, those skilled in the art will recognize that the present invention may be manifested in a variety of forms other than the specific embodiments described and contemplated herein. Accordingly, departures in form and detail may be made without departing from the scope and spirit of the present invention as described in the appended claims.

1.-22. (canceled)

23. A method for treating a patient, comprising:
introducing a material into a vascular system of a patient, wherein the material is configured to occlude vasculature supplying the patient's gastrointestinal system.

24. The method of claim 23, wherein the material includes a plurality of occlusion beads.

25. The method of claim 24, wherein the occlusion beads include a polymeric material.

26. The method of claim 24, wherein the occlusion beads include at least one of polyethylene, polymethyl methacrylate, anadine butadiene styrene, polycarbonate, polyamide, or pebax.

27. The method of claim 24, wherein the occlusion beads include at least one of ceramic, glass, or metal.

28. The method of claim 23, wherein introducing includes inserting a delivery device into an arterial system of the patient.

29. The method of claim 23, wherein introducing includes inserting a catheter into an arterial arcade of the patient.

30. The method of claim 23, wherein the material is configured to occlude vasculature supplying the patient's intestines.

31. The method of claim 30, wherein the material is configured to occlude vasculature supplying the patient's intestinal villi.

32. A method for treating a patient, comprising:
introducing a material into a vascular system of a patient, wherein the material is configured to inhibit nutrient absorption by an intestine of the patient.

33. The method of claim 32, wherein the material includes a plurality of occlusion beads.

34. The method of claim 33, wherein the occlusion beads are suspended in a solution.

35. The method of claim 32, wherein introducing includes inserting a catheter into a superior mesenteric artery of the patient.

36. The method of claim 32, wherein the material is configured to aggregate in a microvasculature of the patient to inhibit blood supply to the intestine.

37. A method for treating a patient, comprising:
inserting a delivery device into an arterial system of the patient; and
using the delivery device, delivering a material into the arterial system, wherein the material is configured to reduce blood circulation to an intestine of the patient.

38. The method of claim 37, wherein the delivery device is a catheter.

39. The method of claim 37, wherein inserting includes inserting the delivery device into the arterial system via at least one of a femoral, subclavian, or radial artery.

40. The method of claim 37, further comprising positioning a distal end of the delivery device in an ostium of an artery supplying a section of the intestine.

41. The method of claim 37, wherein the material is configured to reduce blood circulation by occluding microvasculature supplying the intestine.

42. The method of claim 37, wherein the material includes a plurality of occlusion beads.
